(19) United States
(12) Patent Application Publication
(10) Pub. No.: US 2006/0154303 A1
(43) Pub. Date: Jul. 13, 2006

(54) Evanescent wave excited fluorescence detection method

(76) Inventors: Toru Myogadani, Yokohama-shi (JP); Youji Ebe, Yokohama-shi (JP); Masao Yamada, Yokohama-shi (JP); Jun Hirabayashi, Tsukuba-shi (JP)

Correspondence Address: TOWNSEND & BANTA;c/o PORTFOLIO IP, PO BOX 52050, MINNEAPOLIS, MN, 55402, US

(21) Appl. No.: 11/331,959

(22) Filed: Jan. 13, 2006

(30) Foreign Application Priority Data
Jan. 13, 2005 (JP) 2005-6298

(51) Int. Cl. G01N 33/53; C12M 1/34

(52) U.S. Cl. 435/7.1; 435/287.2

(57) ABSTRACT

In the case where many interactional substances are immobilized on the front surface side of a light transmitting plate capable of generating evanescent waves on the front surface, and where the fluorescence levels in the interactions between the interactional substances and a fluorescence labeled test substance are detected in a liquid layer according to a conventional method, the detection sensitivity and S/N ratio are low due to the background noise. Furthermore, it is necessary to perform a gel filtration purification step for the purpose of removing an excessive amount of the fluorescence dye used for labeling a test substance before the interactions between the fluorescence labeled test substance and the interactional substances are performed. To solve these problems, this invention proposes the above-mentioned method for detecting the evanescent wave excited fluorescence comprising the step of adding a black colorant, for example, a pigment based colorant mainly containing fine particles such as Bokuju.

### BACKGROUND OF THE INVENTION

The present invention relates to an evanescent wave excited fluorescence detection method, particularly an evanescent wave excited fluorescence detection method, in which many interactional substances are immobilized on the front surface side of a light transmitting plate capable of generating evanescent waves on the front surface, and the fluorescence levels in the interactions between the interactional substances and a fluorescently labeled test substance are detected in a liquid layer.

Known are methods and apparatuses, in which evanescent waves (near-field light) generated on a total light reflection surface and sharply attenuating with the increase of distance are used to excite a fluorescent substance labeling the test substance, and the fluorescence level is detected for measuring the interaction of the test substance, etc.

There are patents documents relate to a sample chip analyzing method or apparatus for analyzing the gene expression modes of cells or vital tissues or analyzing antigen-antibody interactions.

For example, U.S. Pat. No. 6,787,364 describes a method and apparatus in which light is irradiated to a waveguide plate at an end face of the waveguide plate capable of totally reflecting and guiding incident light; the evanescent waves generated when the light is totally reflected are used to excite the fluorescent substance labeling the samples to be analyzed; and the fluorescent images formed are used to analyze the samples to be analyzed.

Furthermore, Japanese Pat. No. JP2003-172701 describes a sample chip analyzing apparatus, comprising first and second moving devices respectively capable of moving a sample chip holding member holding a sample chip having many samples immobilized on a substrate capable of guiding incident light, in the longitudinal direction of the sample chip and in the direction perpendicular to the longitudinal direction; a white light source; a filter member of output side for selecting the light with a wavelength for exciting the fluorescent substance labeling a test sample made to react with the samples of the sample chip; an optical fiber bundle for introducing transmitting light into respective light irradiating members; a filter member of light receiving side for selectively transmitting the light emitted from the excited fluorescent substance labeling the test sample having reacted with the samples of the sample chip, provided to face the sample chip between a pair of the light irradiating members; and a light receiving device for delivering an electric signal in response to the emitted light of each predetermined area the light has transmitted.

In the above-mentioned methods comprising the steps of exciting a fluorescent substance by evanescent waves and detecting its fluorescence, in the case where the interactions between the probes installed on the front surface side of a light transmitting plate capable of generating evanescent waves on the front surface and a fluorescently labeled test substance are going to be directly detected in a liquid layer, the following problems occur.

In the case where the liquid layer exists on the front surface side of the light transmitting plate capable of generating evanescent waves on the front surface, the evanescent waves emerging from the light transmitting plate onto the front surface excite not only the fluorescent substance labeling the test substance having reacted with the probes but also the fluorescent substance labeling the test substance floating in the liquid layer near the light transmitting plate without interacting with the probes, to generate fluorescence, and furthermore, the self fluorescence generated from the test substance per se works as disturbing light, to enhance the light intensity of background noise, for lowering the detection sensitivity and the S/N ratio.

In the conventional techniques including the aforesaid inventions described in said patent documents, no countermeasure against such problems has been taken into account.

Moreover the conventional methods need a gel filtration purification step for the purpose of removing an excessive amount of the fluorescence dye used for labeling the test substance before the interactions between the fluorescently labeled test substance and probes are performed.

The object of the present invention is to solve the above-mentioned problems.

### SUMMARY OF THE INVENTION

To solve the above-mentioned problems, this invention proposes an evanescent wave excited fluorescence detection method, in which many interactional substances are immobilized on the front surface side of a light transmitting plate capable of generating evanescent waves on the front surface, and the fluorescence levels in the interactions between the interactional substances and a fluorescently labeled test substance are detected in a liquid layer, characterized in that a black colorant is added to the liquid layer.

Furthermore, this invention as described above proposes that the black colorant is added after the interactions between the interactional substances and the fluorescently labeled test substance or simultaneously with or before start of the interactions between the interactional substances and the fluorescently labeled test substance.

Still furthermore, in this invention as described above, the black colorant can be a pigment based colorant mainly containing fine particles of, for example, carbon black, and as the pigment based colorant, Bokuju (mainly consisting of soot and glue; also called India ink or China ink) can be used.

Still furthermore, this invention as described above proposes that a liquid layer drying preventive plate incapable of transmitting light is installed above on the front surface side of the light transmitting plate.

According to the invention as described above, since the liquid layer is colored black by the added black colorant, the background noise can be reduced and the S/N ratio can be enhanced.

Therefore, it is possible to omit the gel filtration purification step otherwise necessary for removing the excessive amount of the fluorescence dye used for labeling the test substance before the interactions between the fluorescently labeled test substance and the interactional substances.

In the case where the liquid layer drying preventive plate is installed above on the front surface side of the light transmitting plate, since the liquid layer drying preventive plate is made unable to transmit light, for example, by being colored, the penetration of disturbing light through the liquid layer drying preventive plate can be prevented, and the S/N ratio can be further enhanced.

### BRIEF DESCRIPTION OF THE DRAWING

FIG. 1 is a schematic view typically showing an apparatus for analyzing based on the detection of evanescent wave excited fluorescence, to which this invention is applied.

FIG. 2 is CCD camera images showing experimental results 3.

FIG. 3 is CCD camera images showing experimental results 4 and 5.

FIG. 4 is CCD camera images showing experimental results 6.

### PREFERRED EMBODIMENT OF THE INVENTION

This invention is explained below in detail in reference to the drawings showing examples.

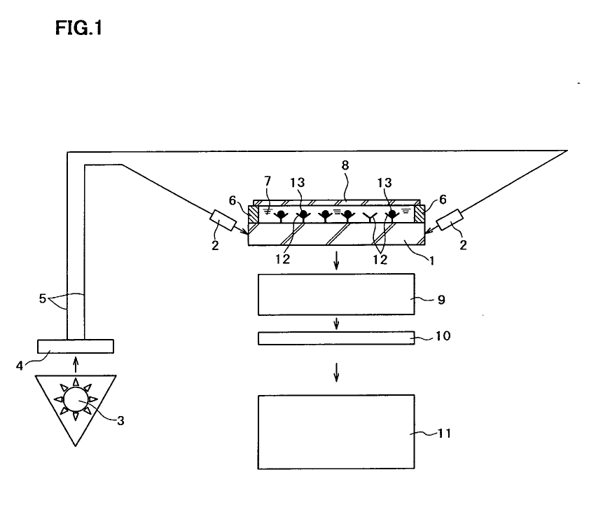

In FIG. 1 symbol 1 denotes a light transmitting plate such as a slide glass, and light irradiating sections 2 are disposed to face the end faces of the light transmitting plate 1. Symbol 3 denotes a white light source, and the white light emitted from the white light source 3 runs through an exciting light filter 4 and falls on optical fibers 5. The light runs through the optical fibers 5 and reaches the light irradiating sections 2.

On the front surface side of the light transmitting plate 1, lateral walls 6 are installed around so that a liquid layer 7 can be held, and above the lateral walls 6, a glass cover 8 as a liquid layer drying preventive plate can be mounted. The glass cover 8 and the lateral walls 6 are made incapable of transmitting light, for example, by being colored black.

On the other hand, on the rear surface side of the light transmitting plate 1, a CCD camera 11 for detecting fluorescence is disposed through an objective lens 9 and a light receiving filter 10.

In the above constitution, many interactional substances 12 are immobilized beforehand on the front surface side of the light transmitting plate 1, and a fluorescently labeled test substance 13 is made to interact with the interactional substances 12 in the liquid layer 7 together with a buffer.

Thus after lapse of a predetermined interaction time, the light transmitting plate 1 is irradiated with exciting light from the light irradiating sections 2, and the luminance values of the fluorescence of the fluorescence label excited by the evanescent waves generated on the front surface side of the light transmitting plate 1 by total reflection are measured on the rear surface side of the light transmitting plate 1 by the CCD camera 11 through the objective lens 9 and the light receiving filter 10. In this invention, in this case, a black colorant is added to the liquid layer 7 for coloring it.

Thus in this invention, the black colorant added blackens the liquid layer, for reducing the background noise and enhancing the S/N ratio. So, experimental results showing the effects of this invention are explained below as examples.

### EXAMPLE 1

As experiments for analyzing sugar chain structure, a microarray having many lectins as interactional substances immobilized on a slide glass used as the light transmitting plate 1 was prepared, and the fluorescence levels in the interactions between a fluorescently labeled glycoprotein and the lectins were detected in a liquid layer using the above-mentioned analyzing apparatus.

Experimental Results 1

At first, 10 vol % of Bokuju was added to a interaction solution containing the fluorescently labeled glycoprotein, and the mixture was supplied onto a slide glass to carry out interactions. In this case, the luminance value of the background where no lectin existed on the slide glass, i.e., the background noise was measured. Furthermore, the luminance value of the portion reflecting the amount of each lectin and the glycoprotein bound to each other was also measured. In this experiment, Bokuju had been added when the interactions between the interactional substances and the fluorescently labeled test substance started.

As described above, in any conventional method, it is necessary to carry out gel filtration purification for the purpose of removing an excessive amount of the fluorescence dye used for labeling the test substance before the interactions between the fluorescently labeled test substance 13 and the interactional substances 12 are performed. So, in this experiment, measurement was made in two cases where the gel filtration purification step was employed and not employed, in combination with additional two cases where Bokuju was added and not added. Table 1 shows the values measured 2 hours after start of the interactions, and Table 2 shows the values measured 20 hours after start of the interactions. The background luminance values stated in Tables 1 and 2 and in the Table 3 described later show background noise values.

TABLE 1

| | | | No gel filtration | | Gel filtration | | Gel filtration | |
|---|---|---|---|---|---|---|---|---|
| | No gel filtration | | purification | | purification performed | | purification performed | |
| | purification | | (10% Bokuju added) | | (conventional condition) | | (10% Bokuju added) | |
| | | S/N | | S/N | | S/N | | S/N |
| | Net intensity* | ratio | Net intensity* | ratio | Net intensity* | ratio | Net intensity* | ratio |
| Lectin 1 | 3836 ± 840 | 1.34 | 113 ± 48 | 1.34 | 953 ± 340 | 1.80 | 105 ± 30 | 1.72 |
| 0.125 mg/ml | | | | | | | | |
| Lectin 1 | 4530 ± 694 | 1.41 | 495 ± 52 | 2.51 | 1829 ± 563 | 2.53 | 537 ± 68 | 4.69 |
| 0.25 mg/ml | | | | | | | | |
| Lectin 1 | 6169 ± 1126 | 1.55 | 2434 ± 1322 | 8.40 | 3638 ± 368 | 4.04 | 2143 ± 250 | 15.74 |
| 0.5 mg/ml | | | | | | | | |
| Lectin 1 | 8800 ± 779 | 1.79 | 3962 ± 354 | 13.05 | 5588 ± 1212 | 5.67 | 3408 ± 263 | 24.45 |
| 1.0 mg/ml | | | | | | | | |
| Lectin 1 | 9072 ± 738 | 1.81 | 3925 ± 160 | 12.94 | 5113 ± 534 | 5.27 | 2852 ± 280 | 20.62 |
| 2.0 mg/ml | | | | | | | | |
| Lectin2 | 1125 ± 1131 | 2.01 | 9257 ± 2684 | 29.16 | 6330 ± 1736 | 6.29 | 7063 ± 729 | 49.59 |
| 0.0625 mg/ml | | | | | | | | |
| Lectin2 | 37274 ± 3959 | 4.34 | 35903 ± 4488 | 110.21 | 30518 ± 4112 | 26.49 | 28494 ± 4041 | 197.03 |
| 0.125 mg/ml | | | | | | | | |
| Lectin2 | 41532 ± 2459 | 4.73 | 41645 ± 5592 | 127.68 | 42448 ± 3978 | 36.45 | 36264 ± 3041 | 250.48 |
| 0.25 mg/ml | | | | | | | | |
| Lectin2 | 49669 ± 2839 | 5.46 | 46047 ± 6184 | 141.07 | 47906 ± 9357 | 41.01 | 38777 ± 8524 | 267. 77 |
| 0.5 mg/ml | | | | | | | | |
| Lectin2 | 41010 ± 2156 | 4.68 | 37811 ± 2930 | 116.02 | 35879 ± 4163 | 30.96 | 32647 ± 2918 | 225.60 |
| 1.0 mg/ml | | | | | | | | |
| Lectin3 | 35341 ± 5177 | 4.17 | 29418 ± 3946 | 90.48 | 38902 ± 6594 | 33.49 | 34138 ± 2284 | 235.85 |
| 0.5 mg/ml | | | | | | | | |
| Lectin4 | 7137 ± 2408 | 1.64 | 3072 ± 1295 | 10.34 | 6734 ± 1176 | 6.62 | 3535 ± 1928 | 25.32 |
| 1 mg/ml | | | | | | | | |

| | | | | |
|---|---|---|---|---|
| Background | 11143 | 329 | 1197 | 145 |
| luminance value | | | | |

*Each net intensity refers to a value obtained by subtracting the background luminance value from the measured luminance value.

TABLE 2

| | | | No gel filtration | | Gel filtration | | Gel filtration | |
|---|---|---|---|---|---|---|---|---|
| | No gel filtration | | purification | | purification performed | | purification performed | |
| | purification | | (10% Bokuju added) | | (conventional condition) | | (10% Bokuju added) | |
| | | S/N | | S/N | | S/N | | S/N |
| | Net intensity* | ratio | Net intensity* | ratio | Net intensity* | ratio | Net intensity* | ratio |
| Lectin 1 | 4868 ± 1151 | 1.29 | 215 ± 56 | 1.27 | 2701 ± 1102 | 2.14 | 121 ± 59 | 1.29 |
| 0.125 mg/ml | | | | | | | | |
| Lectin 1 | 6844 ± 939 | 1.40 | 979 ± 121 | 2.22 | 5616 ± 1635 | 3.37 | 878 ± 98 | 3.09 |
| 0.25 mg/ml | | | | | | | | |
| Lectin 1 | 11538 ± 3688 | 1.68 | 4392 ± 1783 | 6.45 | 9998 ± 1361 | 5.22 | 3905 ± 548 | 10.29 |
| 0.5 mg/ml | | | | | | | | |
| Lectin 1 | 18247 ± 2568 | 2.07 | 7250 ± 584 | 10.00 | 14139 ± 3094 | 6.96 | 6344 ± 445 | 16.09 |
| 1.0 mg/ml | | | | | | | | |
| Lectin 1 | 17325 ± 1673 | 2.02 | 6472 ± 254 | 9.04 | 12859 ± 1263 | 6.42 | 5425 ± 412 | 13.90 |
| 2.0 mg/ml | | | | | | | | |
| Lectin2 | 18167 ± 2402 | 2.07 | 15383 ± 4255 | 20.10 | 11245 ± 2927 | 5.74 | 12054 ± 1113 | 29.67 |
| 0.0625 mg/ml | | | | | | | | |
| Lectin2 | 45097 ± 1906 | 3.65 | 53532 ± 2415 | 67.47 | 46704 ± 4381 | 20.70 | 47575 ± 3052 | 114.17 |
| 0.125 mg/ml | | | | | | | | |
| Lectin2 | 47767 ± 736 | 3.81 | 59725 ± 2992 | 75.16 | 58265 ± 2942 | 25.58 | 57668 ± 2584 | 138.18 |
| 0.25 mg/ml | | | | | | | | |
| Lectin2 | 47517 ± 1243 | 3.79 | 63531 ± 959 | 79.89 | 62495 ± 495 | 27.36 | 63757 ± 1773 | 152.66 |
| 0.5 mg/ml | | | | | | | | |
| Lectin2 | 47977 ± 857 | 3.82 | 57737 ± 1707 | 72.69 | 56184 ± 2630 | 24.70 | 56767 ± 1616 | 136.03 |
| 1.0 mg/ml | | | | | | | | |
| Lectin3 | 46483 ± 1543 | 3.73 | 62165 ± 1517 | 78.19 | 57640 ± 3687 | 25.31 | 56147 ± 2722 | 134.56 |
| 0.5 mg/ml | | | | | | | | |
| Lectin4 | 18868 ± 5943 | 2.11 | 10497 ± 4885 | 14.03 | 18662 ± 2890 | 8.87 | 10367 ± 5116 | 25.66 |
| 1 mg/ml | | | | | | | | |

| | | | | |
|---|---|---|---|---|
| Background | 17004 | 805 | 2371 | 420 |
| luminance value | | | | |

*Each net intensity refers to a value obtained by subtracting the background luminance value from the measured luminance value.

From the measurement results shown in Tables 1 and 2, the following can be seen.

### FOR TABLE 1 (2 Hours After Start of Interactions):

a. In the case where the gel filtration purification step was carried out and where no Bokuju was added, the background noise declined to about 11/100, and in the case where the gel filtration purification step was not carried out and where Bokuju was added, it greatly declined to about 3/100, compared with the case where the gel filtration purification step was not carried out and where no Bokuju was added.

b. In the case where the gel filtration purification step was carried out and where Bokuju was added, the ratio of the luminance value of the portion reflecting the amount of each lectin and the glycoprotein bound to each other to the background noise (S/N ratio) rose to 7.4 times at the maximum (197.03/26.49 when 0.125 mg/mL of lectin 2 was used) compared with the case where the gel filtration purification step was carried out and where no Bokuju was added.

c. In the case where the gel filtration purification step was not carried out and where Bokuju was added, the ratio of the luminance value of the portion reflecting the amount of each lectin and the glycoprotein bound to each other to the background noise (S/N ratio) rose to 27 times at the maximum (127.68/4.73 when 0.25 mg/mL of lectin 2 was used) compared with the case where the gel filtration purification step was not carried out and where no Bokuju was added.

### FOR TABLE 2 (20 Hours After Start of Interactions)

a. In the case where the gel filtration purification step was carried out and where no Bokuju was added, the background noise declined to 14/100, and in the case where the gel filtration purification step was not carried out and where Bokuju was added, it greatly declined to 5/100, compared with the case where the gel filtration purification step was not carried out and where no Bokuju was added.

b. In the case where the gel filtration purification step was carried out and where Bokuju was added, the ratio of the luminance value of the portion reflecting the amount of each lectin and the glycoprotein bound to each other to the background noise (S/N ratio) rose to 5.6 times (152.66/27.36 when 0.5 mg/mL of lectin 2 was used) compared with the case where the gel filtration purification step was carried out and where no Bokuju was added.

c. In the case where the gel filtration purification step was not carried out and where Bokuju was added, the ratio of the luminance value of the portion reflecting the amount of each lectin and the glycoprotein bound to each other to the background noise (S/N ratio) rose to 21 times at the maximum (79.89/3.79 when 0.5 mg/mL of lectin 2 was used) compared with the case where the gel filtration purification step was not carried out and where no Bokuju was added.

From the above-mentioned measurement results, the following can be seen.

a. In both the cases where the gel filtration purification step is carried out and is not carried out, the Bokuju added to the liquid layer can greatly reduce the background noise and enhance the S/N ratio. So, even in the interactions in which the power for binding each lectin and a glycoprotein to each other is low, detection is possible.

b. The reduction of background noise and the enhancement of S/N ratio are remarkable especially in the case where the gel filtration purification is not carried out. So, in this invention, the gel filtration purification step can be omitted. Therefore, the operation can be simplified and measurement in cruder conditions can be made. So, for example, the method of this invention can be applied to a slight amount of a test substance, the purification of which is difficult. That is, the method of this invention can be used for a very wide range of applications.

c. Since weaker binding can be detected as can be seen from the above a and b, for example, the change of luminance with the lapse of time can be more easily measured, and interaction states can be analyzed more accurately in real time.

### Experimental Results 2

Unlike the experiment shown in Tables 1 and 2, in this experiment, Bokuju was not added before start of the interactions between the interactional substances and the fluorescently labeled test substance. Instead 1 to 50 vol %, particularly 1 vol %, 10 vol % or 50 vol % of Bokuju was added to the liquid layer after interactions and immediately before measurement. In this case, the luminance value of the background where no lectin existed on the slide glass, i.e., background noise was measured, and the luminance value of the portion reflecting the amount of each lectin and the glycoprotein bound to each other was also measured. In this experiment, the gel filtration purification step was carried out. The results are shown in Table 3.

TABLE 3

| | | | | | | | | | After standing for 24 | |
|---|---|---|---|---|---|---|---|---|---|---|
| | | | Immediately after | | Immediately after | | Immediately after | | hours subsequently to | |
| | Before addition | | adding 1% Bokuju | | adding 10% Bokuju | | adding 50% Bokuju | | addition of 50% bokuju | |
| | | S/N | | S/N | | S/N | | S/N | | S/N |
| | Net intensity* | ratio | Net intensity* | ratio | Net intensity* | ratio | Net intensity* | ratio | Net intensity* | ratio |
| Lectin I | 40858 ± 1672 | 5.83 | 44202 ± 3998 | 13.28 | 42832 ± 2664 | 13.42 | 41647 ± 3409 | 14.52 | 41375 ± 3302 | 13.29 |
| Lectin II | 27942 ± 1136 | 4.31 | 32802 ± 1587 | 10.12 | 25660 ± 1094 | 8.44 | 22945 ± 925 | 8.45 | 23423 ± 992 | 7.96 |
| LectinIII | 13042 ± 1351 | 2.54 | 14674 ± 518 | 5.08 | 11462 ± 948 | 4.32 | 10169 ± 860 | 4.30 | 10626 ± 1359 | 4.16 |
| LectinIV | 6714 ± 978 | 1.79 | 5910 ± 666 | 2.64 | 5702 ± 961 | 2.65 | 4970 ± 585 | 2.61 | 5940 ± 612 | 2.76 |
| LectinV | 4669 ± 679 | 1.55 | 4050 ± 465 | 2.13 | 3716 ± 479 | 2.08 | 3616 ± 383 | 2.17 | 6062 ± 502 | 2.80 |
| LectinVI | 26129 ± 1169 | 4.09 | 27374 ± 871 | 8.61 | 25904 ± 1282 | 8.51 | 24388 ± 1347 | 8.91 | 24819 ± 1841 | 8.37 |

| | | | | | |
|---|---|---|---|---|---|
| Background | 8452 | 3599 | 3449 | 3082 | 3368 |
| luminance value | | | | | |

*Each net intensity refers to a value obtained by subtracting the background luminance value from the measured luminance value.

From the measurement results shown in Table 3, in the case where Bokuju was added after interactions, the following can be seen.
a. The background noise declined progressively to 43/100, 41/100 and 36/100 respectively at 1%, 10% and 50% of Bokuju, and the S/N ratio was enhanced progressively in relation with it.
b. Even when the added amount of Bokuju was as small as 1%, the effect of reducing the background noise could be obtained.
c. The data obtained after the 50% Bokuju added liquid was allowed to stand for 24 hours showed that the effect of reducing background noise was not adversely affected even after lapse of long time.

### Experimental Results 3

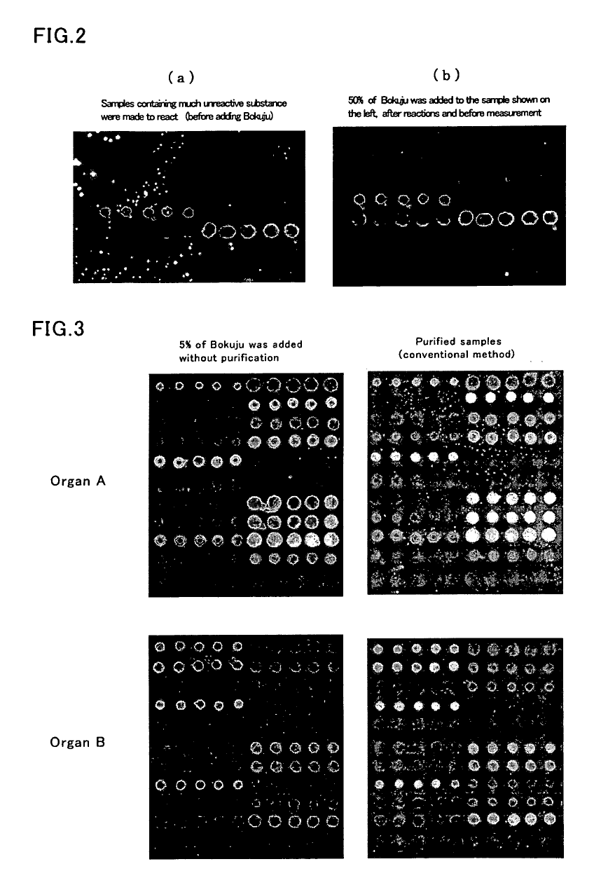

The sample in which the fluorescence dye used for labeling the glycoprotein was non-specifically bound to impurities in the above-mentioned experiment was used, and 50% of Bokuju was added or not added, to obtain images taken by a CCD camera and shown in FIG. 2. Photo (a) shows the case where no Bokuju was added and photo (b), the case where Bokuju was added.

### Experimental Results 4

When samples in which the fluorescent dye was non-specifically bound to impurities were used, bright spots caused by the impurities appeared in addition to the spots of the lectines to be measured, to disturb the measurement, as shown in FIG. 2. However, it can be seen that in the case where Bokuju was added, the noise of these bright spots vanished to allow accurate measurement.

In these experimental results 2 through 4, even in the case where Bokuju was added after interactions, the background noise was reduced while the S/N ratio was enhanced at the time of measurement, for allowing more quantitative analysis of the interactions.

### EXAMPLE 2

### Experimental Results 5

Crude extracts containing the glycoproteins derived from the organs of a mouse were used as samples, and an experiment was carried out using a lectin array immobilized on a slide glass for comprehensively analyzing the sugar chain structure. The results are shown in FIG. 3. In this experiment, the interactions between the lectins and the fluorescently labeled samples were carried out with 5% of Bokuju added and without performing the gel filtration purification step, and on the other hand, the interactions between the lectins and the fluorescently labeled samples were carried out without adding Bokuju and with the gel filtration purification step performed as in any conventional method. The measurement results of the respective cases were compared.

In this experiment, since 5% of Bokuju was added to the samples, the interactions between the lectins and the fluorescently labeled samples, carried out without performing the gel filtration purification step, allowed measurement at sensitivity equivalent or superior to that of purified samples, even though the corresponding interactions carried out without performing the gel filtration purification step in the conventional method allowed little measurement owing to the high background noise. From the result, it can be seen that if this invention is applied, a crude extract containing a slight amount of a vital sample can be analyzed at a high throughput.

### EXAMPLE 3

### Experimental Results 6

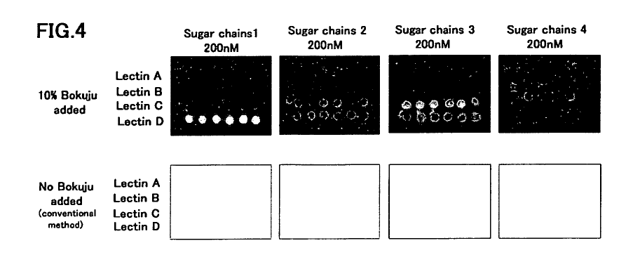

The analyzing apparatus shown in FIG. 1 was used while a interaction solution containing sugar chains was used as the fluorescently labeled test substance, and the interactions between the test substance and lectins were measured in a liquid layer. The results are shown in FIG. 4. Fluorescently labeled sugar chains tend to be very low in detection sensitivity compared with fluorescently labeled glycoproteins, and in any conventional method, even if the gel filtration purification step is performed, detection is difficult. So, for quantitative measurement, the interaction solution must contain the sugar chains at a high concentration, but in this case, the background noise also rises at the same time. So, measurement is impossible.

In the results of this experiment, in a interaction solution containing sugar chains at a high concentration of 200 nM, since the luminance values exceeded the highest measurable value in the conventional method, the obtained images became pure white, not allowing spots to be confirmed. However, when a interaction solution obtained by adding 10 vol % of Bokuju was used, the luminance value reflecting the amount of each lectin and sugar chains bound to each other could be measured. From the experimental results, it can be seen that the analysis over time using sugar chains with low sensitivity also becomes easy if Bokuju is added to a highly concentrated interaction solution.

### INDUSTRIAL APPLICABILITY

According to this invention, as described above, many interactional substances are immobilized on the front surface side of a light transmitting plate capable of generating evanescent waves on the front surface, and the fluorescence levels in the interactions between the interactional substances and a fluorescently labeled test substance are detected in a liquid layer, and in this method, a black colorant is added to the liquid layer. So, the background noise can be reduced and the S/N ratio can be enhanced.

Therefore, it is not required to perform the gel filtration purification step otherwise necessary for removing an excess amount of the fluorescence dye used for labeling a test substance before the interactions between the fluorescently labeled test substance and interactional substances are performed, and the process can be simplified.

Moreover, because of the above, the operation can be simplified and measurement in crude conditions can be made. So, for example, the method of this invention can be applied to a slight amount of a test substance, the purification of which is difficult. That is, the method of this invention can be used for a very wide range of applications.

Still furthermore, in the case where the liquid layer drying preventive plate is installed above on the front surface side of the light transmitting plate, since the liquid layer drying preventive plate is made unable to transmit light, for example, by being colored, the penetration of disturbing light through the liquid layer drying preventive plate can be prevented, and the S/N ratio can be further enhanced.

In view of the above, the applications of this invention include the measurement using a slight amount of a vital sample, the analysis of a sample containing sugar chains, etc. Considering these and other applications, this invention is highly industrially applicable.

The present disclosure relates to subject matter contained in priority Japanese Patent Application No. 2005-6298 filed on Jan. 13, 2005, the contents of which is herein expressly incorporated by reference in its entirety.

1. An evanescent wave excited fluorescence detection method, in which many interactional substances are immobilized on the front surface side of a light transmitting plate capable of generating evanescent waves on the front surface, and the fluorescence levels in the interactions between the interactional substances and a fluorescently labeled test substance are detected in a liquid layer, characterized in that a black colorant is added to the liquid layer.

2. An evanescent wave excited fluorescence detection method, according to claim 1, wherein the black colorant is added after the interactions between the interactional substances and the fluorescently labeled test substance.

3. An evanescent wave excited fluorescence detection method, according to claim 1, wherein the black colorant is added simultaneously with or before start of the interactions between the interactional substances and the fluorescently labeled test substance.

4. An evanescent wave excited fluorescence detection method, according to claim 1, wherein the black colorant is a pigment based colorant.

5. An evanescent wave excited fluorescence detection method, according to claim 4, wherein the pigment based colorant is Bokuju (mainly consisting of soot and glue; also called India ink or China ink).

6. An evanescent wave excited fluorescence detection method, according to claim 1, wherein a liquid layer drying preventive plate incapable of transmitting light is installed above on the front surface side of the light transmitting plate.

7. An evanescent wave excited fluorescence detection method, according to claim 2, wherein the black colorant is a pigment based colorant.

8. An evanescent wave excited fluorescence detection method, according to claim 3, wherein the black colorant is a pigment based colorant.
